(19) United States
(12) Patent Application Publication
(10) Pub. No.: US 2020/0146591 A1
(43) Pub. Date: May 14, 2020

(54) DEVICE WITH FLOW RATE INDICATOR

(71) Applicant: CLEMENT CLARKE INTERNATIONAL LTD., Harlow, Essex (GB)

(72) Inventors: David SPENCER, Harlow, Essex (GB); Ronald BRUIN, Harlow, Essex (GB); Mark SANDERS, Dunstable, Bedfordshire (GB)

(73) Assignee: CLEMENT CLARKE INTERNATIONAL LTD., Harlow, Essex (GB)

(21) Appl. No.: 16/617,135

(22) PCT Filed: May 30, 2018
(86) PCT No.: PCT/EP2018/064200

§ 371 Date: Nov. 26, 2019

(30) Foreign Application Priority Data
May 30, 2017 (GB) 1708602.6

(51) Int. Cl. A61B 5/091; A61B 5/00

(52) U.S. Cl. CPC A61B 5/091; A61B 5/746; A61B 5/682; A61B 5/4542

(57) ABSTRACT

The present invention provides a device for alternatively indicating at least a first fluid flow rate and a second fluid flow rate. The device, which may be a spirometer (e.g. a peak flow meter) comprises an aperture, a mouthpiece and a body defining a fluid flow path extending between the aperture and the mouthpiece. The body comprises a fluid flow rate indicator operable to alternatively generate at least a first sound signal and a second sound signal indicative of the first or second fluid flow rate in a first direction. The fluid flow rate indicator comprises a corrugated portion having a plurality of corrugations extending into the fluid flow path.

### FIELD OF THE INVENTION

The present invention relates to a device for indicating a fluid flow rate. In particular, the present invention relates to a device for indicating an air flow rate during inhalation and/or exhalation. For example, the present invention relates to spirometers for measurement of lung volume and/or peak inspiratory/expiratory flow. The invention also relates to methods of operation of such devices.

### BACKGROUND OF THE INVENTION

There are many devices such as respiratory inhalers (e.g. pressurised metered dose inhalers (pMDIs) and dry powder inhalers (DPIs)) for respiratory drug delivery, spacers/holding chambers for use with such respiratory inhalers and spirometers for measurement of lung volume and/or peak inspiratory/expiratory flow where it is desirable to provide an indication of a fluid (air) flow rate through the device to monitor and/or facilitate correct usage of the device.

Peak flow meters are well known and are typically used to measure a patient's ability to exhale forcibly. This is used to provide an indication of respiratory impairment such as asthma where a patient's airways become narrowed and their ability to forcibly exhale is diminished i.e. the maximum respiratory flow rate at which they are capable of exhaling is reduced. The peak expiratory flow meter can be used for diagnosis and self-management of asthma.

GB-A-2372704 discloses a device for providing an indication of the inspiratory flow rate of a patient. The device includes two reeds adapted to generate an audible signal at different air flow speeds through the device. The first reed generates an audible signal of a first pitch when the air flow reaches a predetermined minimum. The second reed generates an audible signal of a second pitch when the air flow reaches a predetermined maximum. Thus, the patient is informed when the air flow is within a desirable range, between the predetermined minimum and maximum.

Lavorini et al (2010) [F. Lavorini, M. L. Levy, C. Corrigan and G. Crompton, “The ADMIT series—issues in inhalation therapy. 6) Training tools for inhalation devices” Primary Care Respiratory Journal (2010) 19(4) 335-341] set out a review of training tools for inhalation devices, including the device disclosed in GB-A-2372704, referred to as the “2Tone” trainer.

Lavorini et al (2010) comment that two of the most critical patient errors in the uses of pMDI devices are a failure to coordinate inhalation with actuation of the device and inhaling the aerosolized drug too quickly. This is considered to be a critical issue—incorrect use of a pMDI device means that the drug delivered to the patient is being delivered sub-optimally. In turn, this means that the patient does not receive the correct dose of the drug, which can lead to serious problems in the ongoing treatment of conditions such as asthma.

GB-A-2490770 discloses a pMDI actuator body and a spacer for a pMDI inhaler that incorporates an air flow rate indicator comprising a reed which oscillates and generates a sound signal at a predetermined minimum level suitable for delivery of the drug to the patient.

There is a desire to provide an improved air flow rate indicator for devices (e.g. spirometers) that has a simple construction thus facilitating manufacture and reducing manufacturing costs.

### SUMMARY OF THE INVENTION

In a first aspect, the present invention provides a device for indicating a fluid flow rate, the device comprising:

an aperture;

a mouthpiece; and

a body defining a tubular fluid flow path extending between the aperture and the mouthpiece, the body comprising a fluid flow rate indicator operable to generate a sound signal indicative of the fluid flow rate along the fluid flow path,

wherein the fluid flow rate indicator comprises a corrugated portion having at least one corrugation extending into the fluid flow path, and wherein the tubular fluid flow path has a diameter greater than 8 mm.

The inventors have found that providing a fluid flow rate indicator comprising a corrugated portion having at least one and preferably a plurality of corrugations extending into the fluid flow path induces turbulent flow in a fluid moving along the fluid flow path when the fluid flow rate is above a certain minimum rate. The turbulent flow produced generates the sound signal which can provide an indication that the minimum flow rate has been achieved.

Without wishing to be bound to any theory, the inventors believe that the body allows laminar flow of fluid (e.g. gas/air) along the fluid flow path between the aperture and the mouthpiece at low fluid flow rates. As the fluid flow rate increases, the peak(s) and trough(s) of the corrugated portion induce turbulent eddies in the fluid until sound oscillations are generated which match the resonant frequency of the corrugated portion of the body and thus generate a sound signal (which may or may not be audible to the human ear). The sound signal has a narrow frequency and detection of this frequency sound signal (either by the human ear and/or through software for audible sound signals, or through software for non-audible sound signals) can provide a clear indication of the fluid flow rate along the fluid flow path.

Optional features of the invention will now be set out. These are applicable singly or in any combination with any aspect of the invention.

The body may comprise a substantially tubular (e.g. cylindrical) portion defining the substantially tubular (e.g. cylindrical) fluid flow path and/or the body may comprise a substantially tubular (e.g. cylindrical) channel defining the substantially tubular (e.g. cylindrical) fluid flow path. The cross sectional profile of the tubular flow path/tubular body portion/tubular channel may be substantially circular, oval or barrel-shaped.

In some embodiments, the body or body portion may be at least partly formed of plastics material such as polypropylene, acrylonitrile-butadiene-styrene (ABS) copolymer or polycarbonate.

The corrugated portion may form at least part of an inner wall of the body e.g. it may form at least part of (or even the whole of) the body/tubular body portion and/or at least part of the inner wall of the tubular channel. The corrugated portion may be integrally formed as part of the body e.g. it may be integrally formed with the tubular body portion/walls of the tubular channel. For example, the corrugation(s) may be formed (e.g. moulded) on an interior surface of the body/tubular body portion/tubular channel. By providing a corrugated fluid flow rate indicator integrally formed with the body, e.g. formed/moulded on an interior surface, the device has a simple construction with minimal components and no moving parts.

Alternatively, the corrugated portion may be separately formed and inserted into the body e.g. as an inner sleeve at least partially lining the interior surface of the body/tubular body portion/channel or as a strip affixed to the interior surface of the body/tubular body portion/channel.

In some embodiments, the tubular body portion is substantially cylindrical with the corrugated portion provided within an axially oriented recess (extending parallel to the fluid flow path) provided in the tubular body portion.

The inner walls of the channel/inner surface of the body may be substantially smooth (uncorrugated) in areas other than in the corrugated portion. For example, the tubular body portion may have a smooth (uncorrugated) inner surface with the corrugated portion provided within the axially oriented recess.

The body and/or the corrugated portion may be substantially rigid, unlike the known rubbery, flexible corrugated breathing hoses.

In some embodiments, the corrugated portion may completely encircle the fluid flow path. In other embodiments, the corrugated portion may only partially surround the fluid flow path.

In some embodiments, the corrugated portion may extend the entire axial length of the body. In other embodiments, the corrugated portion may extend along a portion of the axial length of the body.

In some embodiments, the corrugated portion may have an axial length (extending parallel to the axis of the fluid flow path) of between 2 and 300 mm, for example between 100 and 300 mm, e.g. between 50 and 300 mm such as around 100 or 150 mm.

The inventors have found that providing a corrugated portion having an axial length of approximately 100 mm allows a plurality of resonant frequencies to be achieved within the body. Each resonant frequency will be associated with a particular flow rate such that a number of particular fluid flow rates can be detected using a single device. In these embodiments, the fluid flow rate indicator is operable to alternatively generate at least a first sound signal and a second sound signal (and preferably further sound signals) to indicate when the fluid flow rate in a first direction along the fluid flow path is at the first or second (or further) fluid flow rate. The first direction may be from the aperture to the mouthpiece or from the mouthpiece to the aperture.

The frequency of the sound signal generated may be detected by ear by the patient (a lower frequency being observed as a lower tone/note) or the patient may be provided with software (e.g. in the form of a mobile phone app) to detect the frequency of the sound signal. The software may be adapted to provide feedback to a remote location. Furthermore, the software may be adapted to provide a visual indication (e.g. a colour coded indication) of the frequency generated during use.

The tubular body portion/channel has an internal diameter of greater than 8 mm e.g. equal to or greater than 9 mm, equal or greater than 10 mm, equal or greater than 11 mm, equal or greater than 12 mm such as around 12.5 mm, equal or greater than 13 mm e.g. between 13 and 13.5 mm, such as 13.1, 13.2, 13.3, 13.4 or 13.5 m, equal or greater than 14 mm e.g. around 14.5 mm thus providing a tubular air flow path having a diameter equal or greater than 5 mm, e.g. equal or greater than 6 mm, equal or greater than 7 mm, equal or greater than 8 mm, equal to or greater than 9 mm, equal or greater than 10 mm, equal or greater than 11 mm, equal or greater than 12 mm such as around 12.5 mm, equal or greater than 13 mm such as around 13.1 or 13.2 or 13.3 or 13.4 or 13.5 mm or equal or greater than 14 mm such as around 14.5 mm.

In some embodiments, the tubular body portion/channel has an internal diameter up to 20 mm.

In a particularly preferred embodiments the internal dimeter is around 13 mm (preferably) 13.085 mm) and the axial length of the corrugated portion is around 100 mm. This has been found to provide a device giving a different sound signal approximately every 50 L/min.

In some embodiments, the resistance of the tubular body portion/channel is between 0.3 and 3.6 kPa at a flow rate of 30 L/min and between 1.7 and 18.5 kPa at a flow rate of 60 L/min.

The corrugated portion may comprise a plurality of parallel ridges/peaks spaced by a plurality of troughs/furrows which at least partially encircle the fluid flow path (and which may be formed into the inner surface of the body portion/walls of the channel).

The plurality of ridges/troughs (or the single ridge/trough for the single corrugation) may be oriented substantially perpendicularly to the fluid flow path or they/it may be at an angle to the fluid flow path.

In other embodiments, the corrugated portion comprises at least one spiral or screw-thread ridge/peak which encircles the fluid flow path (and which may be formed on the interior surface of the body/walls of the channel).

In some embodiments, the corrugated portion comprises between 1 and 170 corrugations, for example, it may comprise between 2 and 100 corrugations, e.g. between 2 and 30 corrugations or between 2 and 10 corrugations.

The pitch of the corrugations i.e. the spacing between adjacent peaks may be between 2-5 mm e.g. around 3 mm.

The height of the corrugation(s) i.e. the height from the base of a trough to the apex of the peak may be between 0.5 and 2.0 mm, for example between 0.5 and 1.0 mm e.g. around 0.6 mm.

In some embodiments, the or each ridge in the corrugated portion has an unsymmetrical longitudinal cross-sectional profile (i.e. the cross-sectional profile parallel to the direction of fluid flow). For example, the or each ridge may have a substantially sawtooth/shark fin profile with differing gradients on opposing (upstream/downstream) sides. The apex of the or each ridge is preferably rounded.

By providing an asymmetrical ridge, the device can be used to produce an inhalation sound signal when fluid flows from the aperture to the mouthpiece (e.g. during inhalation) and an exhalation sound signal when fluid flows from the mouthpiece to the aperture (e.g. during exhalation). The inhalation and exhalation sound signals could have the same frequency. In this way, two identical sound signals could be generated, one at the first (inhalation) flow rate along the flow path from the aperture to the mouthpiece and one at the (same) second (exhalation) flow rate along the flow path from the mouthpiece to the aperture.

In other embodiments, the inhalation and exhalation sound signals may have a different frequency. In this way, the inhalation sound signal could be generated at the first (inhalation) flow rate along the flow path from the aperture to the mouthpiece and the exhalation sound signal could be generated at a (different) second (exhalation) flow rate along the flow path from the mouthpiece to the aperture.

In some embodiments, the corrugated portion extends to the aperture. In other embodiments, the corrugated portion is spaced from the aperture.

In preferred embodiments, the corrugated portion comprises a lead-in portion at its axial end the lead-in portion comprising the or one of the ridges such that as fluid first enters the corrugated portion it enters on a “rising-slope” and is directed towards the axis of the body/channel by the inclined surface of the or one of the ridges.

Some embodiments comprise a plurality of corrugated portions as described above. The corrugated portions may be axially spaced along the tubular body portion/channel with the un-corrugated e.g. smooth inner surface of the tubular body portion/channel interposed between the corrugated portions. Alternatively, they may be circumferentially spaced around the tubular body portion/channel.

In some embodiments, the body may have a substantially smooth outer surface (opposing the inner surface which defines the fluid flow path). In other embodiments, the body may have a corrugated outer surface (e.g. opposing the corrugated portion in the fluid flow path) for providing a visual and tactile distinction to users over known devices without the corrugated flow rate indicator.

In some embodiments, the device is a patient inhalation/exhalation device such as a spirometer e.g. a peak flow meter for measuring air flow rate during exhalation/inhalation by a patient.

In these embodiments, the present invention provides a patient inhalation/exhalation device (such as a spirometer/peak flow meter) comprising:
at least one aperture for inlet or outlet of air into/from the device;
a mouthpiece for communication with the mouth of the patient;
a body defining a tubular air flow path extending between the aperture and the mouthpiece along which air is drawn to the mouthpiece by inhalation by the patient or air is forced towards the aperture by exhalation by the patient, the body comprising an air flow rate indicator operable to generate a sound signal indicative of the air flow rate along the air flow path,
wherein the air flow rate indicator comprises a corrugated portion having at least one corrugation extending into the fluid flow path, and wherein the tubular fluid flow path has a diameter greater than 8 mm.

Such a spirometer/peak flow meter has no moving parts which complicate manufacture and which may wear out. Furthermore, such a spirometer would not require periodic calibration.

The corrugated portion and body may be as described above and there may be a plurality of corrugated portions. In some embodiments, the body may be at least partly formed of plastics material such as polypropylene, acrylonitrile-butadiene-styrene (ABS) copolymer or polycarbonate.

The device is preferably adapted such that the sound signal is generated at an air flow rate of between 30 and 800 L/min.

In these embodiments, the corrugated portion may have an axial length (extending parallel to the axis of the fluid flow path) of between 50 and 300 mm. Such a corrugated portion may have between 1 and 170 corrugations, for example, it may comprise between 2 and 100 corrugations, e.g. between 2 and 30 corrugations or between 2 and 10 corrugations.

The present inventors have found that using a corrugated portion having this axial length provides a plurality of possible resonant frequencies within the body and the frequency of resonance established can provide an indication of the air flow rate through the device (and thus the force of exhalation/inhalation by the patient). An exacerbation of a respiratory condition such as asthma will result in the patient only being able to generate a lower frequency resonance. This reduction in frequency (which can be detected audibly or electronically) can alert the patient to the need to take appropriate action such as increasing medication or seeking medical assistance. In these embodiments, the fluid flow rate indicator is operable to alternatively generate at least a first sound signal and a second sound signal (and preferably further sound signals) to indicate when the fluid flow rate in a first direction along the fluid flow path is at the first or second (or further) fluid flow rate. The first direction may be from the aperture to the mouthpiece (an inhalation direction) or from the mouthpiece to the aperture (an exhalation direction).

A patient may typically be able to generate the first sound signal at a first frequency. An exacerbation of a respiratory condition such as asthma will result in the patient only being able to generate a lower frequency resonance (which results in the second sound signal instead of the first). This reduction in frequency (which can be detected audibly or using software) can alert the patient to the need to take appropriate action such as increasing medication or seeking medical assistance.

The frequency of the sound signal generated may be detected by ear by the patient (a lower frequency being observed as a lower tone/note) or the patient may be provided with software (e.g. in the form of a mobile phone app) to detect the frequency of the sound signal. The software may be adapted to provide feedback to a healthcare provider to assist in management of the respiratory condition. Furthermore, the software may be adapted to provide a visual indication (e.g. a colour coding) of the frequency generated by the patient.

In the spirometer, the mouthpiece and the body may be substantially co-axial. The mouthpiece and body may be substantially tubular e.g. cylindrical.

The spirometer device is preferably adapted such that the sound signal is generated at an air flow rate of between 30 and 800 L/min.

In a second aspect, the present invention provides a device for alternatively indicating at least a first fluid flow rate and a second fluid flow rate, the device comprising:
an aperture;
a mouthpiece; and
a body defining a fluid flow path extending between the aperture and the mouthpiece, the body comprising a fluid flow rate indicator operable to alternatively generate at least a first sound signal and a second sound signal to indicate when the fluid flow rate in a first direction along the fluid flow path is at the first or second fluid flow rate,
wherein the fluid flow rate indicator comprises a corrugated portion having a plurality of corrugations extending into the fluid flow path.

The first direction may be from the aperture to the mouthpiece or from the mouthpiece to the aperture.

The present inventors have found that using a corrugated portion having a plurality of corrugations provides a plurality of (at least two) possible distinct resonant frequencies within the body and the frequency of resonance established can provide an indication of the air flow rate through the device.

The frequencies of the sound signals may be detected by ear (a lower frequency being observed as a lower tone/note) or the user may be provided with software (e.g. in the form of a mobile phone app) to detect the frequency of the sound signal. The software may be adapted to provide feedback to a remote location. Furthermore, the software may be adapted to provide a visual indication (e.g. a colour coded indication) of the frequency generated.

In some embodiments, the air flow rate indicator is operable to alternatively generate at least three, four, five, six, seven, eight, nine, ten, eleven, twelve or thirteen or more sound signals to indicate when the air flow rate along the air flow path is at a first, second, third, fourth, fifth, sixth, seventh, eighth, ninth, tenth, eleventh, twelfth, thirteenth, etc. level.

The body may comprise a substantially tubular (e.g. cylindrical) portion defining the substantially tubular (e.g. cylindrical) fluid flow path and/or the body may comprise a substantially tubular (e.g. cylindrical) channel defining the substantially tubular (e.g. cylindrical) fluid flow path. The cross sectional profile of the tubular flow path/tubular body portion/tubular channel may be substantially circular, oval or barrel-shaped.

In some embodiments, the body or body portion may be at least partly formed of plastics material such as polypropylene, acrylonitrile-butadiene-styrene (ABS) copolymer or polycarbonate.

The corrugated portion may form at least part of an inner wall of the body e.g. it may form at least part of (or even the whole of) the body/tubular body portion and/or at least part of the inner wall of the tubular channel. The corrugated portion may be integrally formed as part of the body e.g. it may be integrally formed with the tubular body portion/walls of the tubular channel. For example, the corrugation(s) may be formed (e.g. moulded) on an interior surface of the body/tubular body portion/tubular channel. By providing a corrugated fluid flow rate indicator integrally formed with the body, e.g. formed/moulded on an interior surface, the device has a simple construction with minimal components and no moving parts.

Alternatively, the corrugated portion may be separately formed and inserted into the body e.g. as an inner sleeve at least partially lining the interior surface of the body/tubular body portion/channel or as a strip affixed to the interior surface of the body/tubular body portion/channel.

In some embodiments, the tubular body portion is substantially cylindrical with the corrugated portion provided within an axially oriented recess (extending parallel to the fluid flow path) provided in the tubular body portion.

The inner walls of the channel/inner surface of the body may be substantially smooth (uncorrugated) in areas other than in the corrugated portion. For example, the tubular body portion may have smooth (un-corrugated) inner surface with the corrugated portion provided within the axially oriented recess.

The body and/or the corrugated portion may be substantially rigid, unlike the known rubbery, flexible corrugated breathing hoses.

In some embodiments, the corrugated portion may completely encircle the fluid flow path. In other embodiments, the corrugated portion may only partially surround the fluid flow path.

In some embodiments, the corrugated portion may extend the entire axial length of the body.

In other embodiments, the corrugated portion may extend along a portion of the axial length of the body.

In some embodiments, the corrugated portion may have an axial length (extending parallel to the axis of the fluid flow path) of between 2 and 300 mm, for example between 100 and 300 mm, e.g. between 50 and 300 mm such as around 100 or 150 mm.

The tubular body portion/channel has an internal diameter of equal or greater than 5 mm, e.g. equal or greater than 6 mm, equal or greater than 7 mm, equal or greater than 8 mm, equal to or greater than 9 mm, equal or greater than 10 mm, equal or greater than 11 mm, equal or greater than 12 mm such as around 12.5 mm, equal or greater than 13 mm such as between 13 and 13.5 mm e.g. around 13.1, 13.2, 13.3, 13.4 or 13.5 mm, equal or greater than 14 mm such as around 14.5 mm thus providing a tubular air flow path having a diameter equal or greater than 5 mm, e.g. equal or greater than 6 mm, equal or greater than 7 mm, equal or greater than 8 mm, equal to or greater than 9 mm, equal or greater than 10 mm, equal or greater than 11 mm, equal or greater than 12 mm such as around 12.5 mm, equal or greater than 13 mm such as around 13.1 or 13.2 or 13.3 or 13.4 or 13.5 mm or equal or greater than 14 mm such as around 14.5 mm.

In some embodiments, the tubular body portion/channel has an internal diameter up to 20 mm.

In a particularly preferred embodiments the internal dimeter is around 13 mm (preferably) 13.085 mm) and the axial length of the corrugated portion is around 100 mm. This has been found to provide a device giving a different sound signal every 50 L/min.

In some embodiments, the resistance of the tubular body portion/channel is between 0.3 and 3.6 kPa at a flow rate of 30 L/min and between 1.7 and 18.5 kPa at a flow rate of 60 L/min.

The corrugated portion may comprise a plurality of parallel ridges/peaks spaced by a plurality of troughs/furrows which at least partially encircle the fluid flow path (and which may be formed into the inner surface of the body portion/walls of the channel).

The plurality of ridges/troughs (or the single ridge/trough for the single corrugation) may be oriented substantially perpendicularly to the fluid flow path or they/it may be at an angle to the fluid flow path.

In other embodiments, the corrugated portion comprises at least one spiral or screw-thread ridge/peak which encircles the fluid flow path (and which may be formed on the interior surface of the body/walls of the channel).

In some embodiments, the corrugated portion comprises between 2 and 170 corrugations, for example, it may comprise between 2 and 100 corrugations, or it may comprise between 2-30 corrugations or 2-10 corrugations.

The pitch of the corrugations i.e. the spacing between adjacent peaks may be between 2-5 mm e.g. around 3 mm.

The height of the corrugation(s) i.e. the height from the base of a trough to the apex of the peak may be between 0.5 and 2.0 mm, for example between 0.5 and 1.0 mm e.g. around 0.6 mm.

In some embodiments, the or each ridge in the corrugated portion has an unsymmetrical longitudinal cross-sectional profile (i.e. the cross-sectional profile parallel to the direction of fluid flow). For example, the or each ridge may have a substantially sawtooth/shark fin profile with differing gradients on opposing (upstream/downstream) sides. The apex of the or each ridge is preferably rounded.

By providing an asymmetrical ridge, the device can be used to produce an inhalation sound signal when fluid flows from the aperture to the mouthpiece (e.g. during inhalation) and an exhalation sound signal when fluid flows from the mouthpiece to the aperture (e.g. during exhalation). The inhalation and exhalation sound signals could have different frequencies. In this way, two different sound signals could be generated, one at a first (inhalation) flow rate along the flow path from the aperture to the mouthpiece and one at the (same) second (exhalation) flow rate along the flow path from the mouthpiece to the aperture.

In other embodiments, the inhalation and exhalation sound signals may have a different frequency. In this way, the inhalation sound signal could be generated at a first (inhalation) flow rate along the flow path from the aperture to the mouthpiece and the exhalation sound signal could be generated at a (different) second (exhalation) flow rate along the flow path from the mouthpiece to the aperture.

In some embodiments, the corrugated portion extends to the aperture. In other embodiments, the corrugated portion is spaced from the aperture.

In preferred embodiments, the corrugated portion comprises a lead-in portion at its axial end the lead-in portion comprising the or one of the ridges such that as fluid first enters the corrugated portion it enters on a “rising-slope” and is directed towards the axis of the body/channel by the inclined surface of the or one of the ridges.

Some embodiments comprise a plurality of corrugated portions as described above. The corrugated portions may be axially spaced along the tubular body portion/channel with the un-corrugated e.g. smooth inner surface of the tubular body portion/channel interposed between the corrugated portions. Alternatively, they may be circumferentially spaced around the tubular body portion/channel.

In some embodiments, the body may have a substantially smooth outer surface (opposing the inner surface which defines the fluid flow path). In other embodiments, the body may have a corrugated outer surface (e.g. opposing the corrugated portion in the fluid flow path) for providing a visual and tactile distinction to users over known devices without the corrugated flow rate indicator.

In some embodiments, the device is a patient inhalation/exhalation device such as a spirometer e.g. a peak flow meter for measuring air flow rate during exhalation/inhalation by a patient.

In these embodiments, the present invention provides a patient inhalation/exhalation device (such as a spirometer/peak flow meter) comprising:
at least one aperture for inlet or outlet of air into/from the device;
a mouthpiece for communication with the mouth of the patient;
a body defining an air flow path extending between the aperture and the mouthpiece along which air is drawn to the mouthpiece by inhalation by the patient or air is forced towards the aperture by exhalation by the patient, the body comprising an air flow rate indicator operable to alternatively generate at least a first and a second sound signal indicative of the air flow rate along the air flow path in a first direction,
wherein the air flow rate indicator comprises a corrugated portion having a plurality of corrugations extending into the fluid flow path.

The first direction may be from the aperture to the mouthpiece (an inhalation direction) or from the mouthpiece to the aperture (an exhalation direction).

The present inventors have found that using a corrugated portion having a plurality of corrugations provides a plurality of (at least two) possible distinct resonant frequencies within the body and the frequency of resonance established can provide an indication of the air flow rate through the device (and thus the force of exhalation/inhalation by the patient). A patient may typically be able to generate the first sound signal at a first frequency. An exacerbation of a respiratory condition such as asthma will result in the patient only being able to generate a lower frequency resonance (which results in the second sound signal instead of the first), this reduction in frequency (which can be detected audibly or using software) can alert the patient to the need to take appropriate action such as increasing medication or seeking medical assistance.

The frequency of the sound signals may be detected by ear by the patient (a lower frequency being observed as a lower tone/note) or the patient may be provided with software (e.g. in the form of a mobile phone app) to detect the frequency of the sound signal. The software may be adapted to provide feedback to a healthcare provider to assist in management of the respiratory condition. Furthermore, the software may be adapted to provide a visual indication (e.g. a colour coded indication) of the frequency generated by the patient.

Such a spirometer/peak flow meter has no moving parts which complicate manufacture and which may wear out. Furthermore, such a spirometer would not require periodic calibration.

The corrugated portion and body may be as described above and there may be a plurality of corrugated portions. In some embodiments, the body may be at least partly formed of plastics material such as polypropylene, acrylonitrile-butadiene-styrene (ABS) copolymer or polycarbonate.

The spirometer/peak flow meter is preferably adapted such that the sound signal is generated at an air flow rate of between 30 and 800 L/min.

In some embodiments, the corrugated portion may have an axial length (extending parallel to the axis of the fluid flow path) of between 2 and 300 mm, for example between 100 and 300 mm, e.g. between 50 and 300 mm such as around 100 or 150 mm.

The corrugated portion may have an axial length (extending parallel to the axis of the fluid flow path) of between 100 and 300 mm.

Such a corrugated portion may comprise between 2 and 170 corrugations, for example, it may comprise between 2 and 100 corrugations, or it may comprise between 2-30 corrugations or 2-10 corrugations.

The tubular body portion/channel has an internal diameter of equal or greater than 5 mm, e.g. equal or greater than 6 mm, equal or greater than 7 mm, equal or greater than 8 mm, equal to or greater than 9 mm, equal or greater than 10 mm, equal or greater than 11 mm, equal or greater than 12 mm such as around 12.5 mm, equal or greater than 13 mm e.g. between 13 and 13.5 mm, such as 13.1, 13.2, 13.3, 13.4 or 13.5 mm, equal or greater than 14 mm e.g. 14.5 mm thus providing a tubular air flow path having a diameter equal or greater than 5 mm, e.g. equal or greater than 6 mm, equal or greater than 7 mm, equal or greater than 8 mm, equal to or greater than 9 mm, equal or greater than 10 mm, equal or greater than 11 mm, equal or greater than 12 mm such as around 12.5 mm, equal or greater than 13 mm such as around 13.1 or 13.2 or 13.3 or 13.4 or 13.5 mm or equal or greater than 14 mm such as around 14.5 mm.

In some embodiments, the tubular body portion/channel has an internal diameter up to 20 mm.

In a particularly preferred embodiments the internal dimeter is around 13 mm (preferably) 13.085 mm) and the axial length of the corrugated portion is around 100 mm. This has been found to provide a device giving a different sound signal every 50 L/min.

In the spirometer, the mouthpiece and the body may be substantially co-axial. The mouthpiece and body may be substantially tubular e.g. cylindrical.

In a third aspect, the present invention provides a device according to the first aspect and a sound receiver for detecting a sound signal.

In a fourth aspect, the present invention provides a device according to the second aspect and a sound receiver for detecting first and second sound signals.

In some embodiments, the sound receiver comprises computer software e.g. an application for running on a mobile device such as a smartphone app. The FrequenSee™ app, available as an Apple® and Android® app, may be used for detecting the sound signal(s). The software may be adapted to provide feedback to a remote location such as healthcare provider to assist in management of the respiratory condition. Furthermore, the software may be adapted to provide a visual indication (e.g. a colour coded indication) of the frequency generated by the patient.

In any embodiment of any of the above described aspects, the mouthpiece may have a wider internal diameter than the internal diameter of the tubular body portion/channel. It be have an oval or barrel shaped internal bore. It may be formed of the same plastics material as the device. It may be integral with the device or it may be separate and connectable to the device e.g. using an interference e.g. a push fit connection.

In a fifth aspect, the present invention provides a method of monitoring peak expiratory or inhalatory flow, the method comprising:
providing a system according to the third or fourth aspect,
exhaling or inhaling through the mouthpiece of the device;
detecting the sound signal generated.

In some embodiments, the method comprises recording (e.g. using computer software such as an application for running on a mobile device such as a smartphone app) the frequency of the sound signal and comparing to a reference frequency, the reference frequency having being generated previously by the patient. The method may provide providing an alert (e.g. a visual or audible alert) to the patient if the frequency of the sound signal is below the reference frequency. The method may comprise proving feedback to a remote location (e.g. a clinician).

This information can be used to monitor exhalation/inhalation capability of by the patient. It can be used (either by the patient or by a healthcare provider) to ensure that appropriate action is taken in the event of a decrease in lung function.

In a sixth aspect, the present invention provides use of a device according to the first or second aspect to measure lung volume or peak inhalatory/exhalatory flow in a patient.

### BRIEF DESCRIPTION OF FIGURES

FIG. 1 shows the flow rates at the resonant nodes in a 7 mm diameter tube; and

FIG. 2 shows the frequency response measurement with the top trace representing the sound frequencies and the bottom trace being background noise.

### EXPERIMENTAL DETAILS

A corrugated tube having an internal diameter of 7 mm and a corrugated portion comprising 25 corrugations and an axial length of 99 mm was formed of polypropylene. Various flow rates through the corrugated tube were applied and the frequency of the sound signals generated were detected using a frequency monitor.

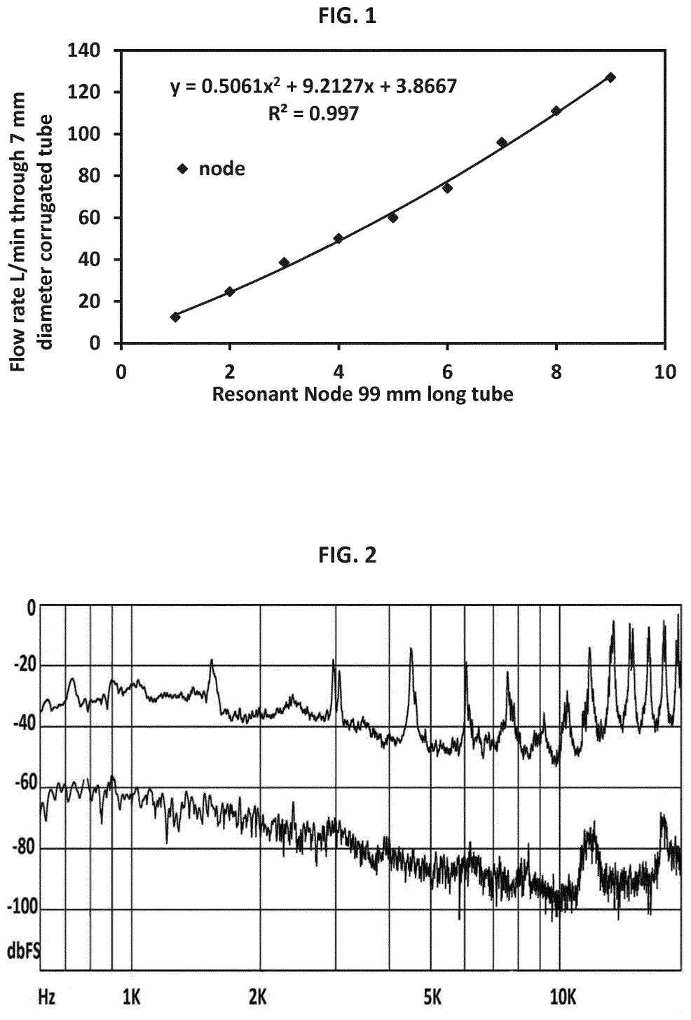

The results are shown in Table 1 below and in FIGS. 1 and 2:

TABLE 1

| Resonant node | Frequency/Hz | Flow rate L/min |
|---|---|---|
| 1 | 1.5 | 12 |
| 2 | 3.0 | 25 |
| 3 | 4.4 | 39 |
| 4 | 6.1 | 50 |
| 5 | 7.6 | 60 |
| 6 | 9.1 | 74 |
| 7 | 10.4 | 96 |
| 8 | 11.8 | 111 |
| 9 | 13.3 | 127 |

It can be seen that the device provides a plurality of resonant nodes with increasing flow rate through the device generating an increasing frequency sound signal.

Adult human respiratory flow typically ranges from 50 to 700 L/min and therefore, whilst the 7 mm diameter device may suitable for measurement of child respiratory flow, larger diameter devices are envisaged for measurement of adult respiratory flow.

Using the data generated using the 7 mm device, the predicted flow rates at various nodes in larger diameter devices were calculated for devices having dimensions shown in Table 2 below:

TABLE 2

| Internal | Axial length of corrugated | Surface area of corrugated |
|---|---|---|
| diameter/mm | portion/mm | portion/m² |
| 8 | 100 | 5.0272 × 10⁻⁵ |
| 9 | 100 | 6.3626 × 10⁻⁵ |
| 12.5 | 100 | 1.23 × 10⁻⁴ |
| 13.085 | 100 | 1.35 × 10⁻⁴ |

The calculations are shown below in Tables 3 (8 mm diameter), 4 (9 mm diameter), 5 (12.5 mm diameter) and 6 (13.085 mm).

TABLE 3
8 mm diameter tube

| | Airspeed across | | | |
|---|---|---|---|---|
| | corrugate to | | | |
| | create sound | | | |
| | based on 7 mm | | Predicted | |
| Resonant | diameter test | | flow rate | Resonant |
| node | results m/sec | Volume m³/s | L/min | frequency Hz |
| 1 | 6.2 | 3.117 × 10⁻⁴ | 19 | 1.5 |
| 2 | 12.4 | 6.234 × 10⁻⁴ | 37 | 2.8 |
| 3 | 18.6 | 9.351 × 10⁻⁴ | 56 | 4.2 |
| 4 | 24.8 | 1.247 × 10⁻³ | 75 | 5.6 |
| 5 | 31.0 | 1.558 × 10⁻³ | 94 | 7.0 |
| 6 | 37.2 | 1.870 × 10⁻³ | 112 | 8.3 |
| 7 | 43.4 | 2.182 × 10⁻³ | 131 | 9.7 |
| 8 | 49.6 | 2.493 × 10⁻³ | 150 | 11.1 |
| 9 | 55.8 | 2.805 × 10⁻³ | 168 | 12.5 |
| 10 | 62.0 | 3.117 × 10⁻³ | 187 | 13.9 |
| 11 | 68.2 | 3.429 × 10⁻³ | 206 | 15.3 |
| 12 | 74.4 | 3.740 × 10⁻³ | 224 | 16.7 |
| 13 | 80.6 | 4.052 × 10⁻³ | 243 | 18.0 |

TABLE 4
9 mm diameter tube

| | Airspeed across | | |
|---|---|---|---|
| | corrugate to | | |
| | create sound | | |
| | based on 7 mm | | |
| Resonant | diameter test | | Predicted flow |
| node | results m/sec | Volume m³/s | rate L/min |
| 1 | 6.2 | 3.945 × 10⁻⁴ | 24 |
| 2 | 12.4 | 7.890 × 10⁻⁴ | 47 |
| 3 | 18.6 | 1.183 × 10⁻³ | 71 |
| 4 | 24.8 | 1.578 × 10⁻³ | 95 |
| 5 | 31.0 | 1.972 × 10⁻³ | 118 |
| 6 | 37.2 | 2.367 × 10⁻³ | 142 |
| 7 | 43.4 | 2.761 × 10⁻³ | 166 |
| 8 | 49.6 | 3.156 × 10⁻³ | 189 |
| 9 | 55.8 | 3.550 × 10⁻³ | 213 |
| 10 | 62.0 | 3.945 × 10⁻³ | 237 |
| 11 | 68.2 | 4.339 × 10⁻³ | 260 |
| 12 | 74.4 | 4.734 × 10⁻³ | 284 |
| 13 | 80.6 | 5.128 × 10⁻³ | 308 |

TABLE 5
12.5 mm diameter tube

| | Airspeed across | | |
|---|---|---|---|
| | corrugate to | | |
| | create sound | | |
| | based on 7 mm | | |
| Resonant | diameter test | | Predicted flow |
| node | results m/sec | Volume m³/s | rate L/min |
| 1 | 6.2 | 7.610 × 10⁻⁴ | 46 |
| 2 | 12.4 | 1.552 × 10⁻³ | 91 |
| 3 | 18.6 | 2.283 × 10⁻³ | 137 |
| 4 | 24.8 | 3.044 × 10⁻³ | 183 |
| 5 | 31.0 | 3.805 × 10⁻³ | 228 |
| 6 | 37.2 | 4.566 × 10⁻³ | 274 |
| 7 | 43.4 | 5.327 × 10⁻³ | 320 |
| 8 | 49.6 | 6.088 × 10⁻³ | 365 |
| 9 | 55.8 | 6.849 × 10⁻³ | 411 |
| 10 | 62.0 | 7.610 × 10⁻³ | 457 |
| 11 | 68.2 | 8.370 × 10⁻³ | 502 |
| 12 | 74.4 | 9.131 × 10⁻³ | 548 |
| 13 | 80.6 | 9.892 × 10⁻³ | 594 |

TABLE 6
13.085 mm diameter tube

| | Airspeed across | | |
|---|---|---|---|
| | corrugate to | | |
| | create sound | | |
| | based on 7 mm | | |
| Resonant | diameter test | | Predicted flow |
| node | results m/sec | Volume m³/s | rate L/min |
| 1 | 6.2 | 8.338 × 10⁻⁴ | 50 |
| 2 | 12.4 | 1.668 × 10⁻³ | 100 |
| 3 | 18.6 | 2.502 × 10⁻³ | 150 |
| 4 | 24.8 | 3.335 × 10⁻³ | 200 |
| 5 | 31.0 | 4.169 × 10⁻³ | 250 |
| 6 | 37.2 | 5.003 × 10⁻³ | 300 |
| 7 | 43.4 | 5.837 × 10⁻³ | 350 |
| 8 | 49.6 | 6.671 × 10⁻³ | 400 |
| 9 | 55.8 | 7.505 × 10⁻³ | 450 |
| 10 | 62.0 | 8.338 × 10⁻³ | 500 |
| 11 | 68.2 | 9.172 × 10⁻³ | 550 |
| 12 | 74.4 | 1.001 × 10⁻² | 600 |
| 13 | 80.6 | 1.084 × 10⁻² | 650 |

It can be seen that as the diameter increases, a device sounding at a wide range of flow rates covering the normal adult respiratory range can be obtained.

While the invention has been described in conjunction with the exemplary embodiments described above, many equivalent modifications and variations will be apparent to those skilled in the art when given this disclosure. Accordingly, the exemplary embodiments of the invention set forth above are considered to be illustrative and not limiting. Various changes to the described embodiments may be made without departing from the scope of the invention as defined in the claims.

1. A device for alternatively indicating at least a first fluid flow rate and a second fluid flow rate, the device comprising:
an aperture;
a mouthpiece; and
a body defining a fluid flow path extending between the aperture and the mouthpiece, the body comprising a fluid flow rate indicator operable to alternatively generate at least a first sound signal and a second sound signal to indicate when the fluid flow rate in a first direction along the fluid flow path is at the first or second fluid flow rate,
wherein the fluid flow rate indicator comprises a corrugated portion having a plurality of corrugations extending into the fluid flow path.

2. A device according to claim 1 wherein the body is at least partly formed of plastics material.

3. A device according to claim 1 wherein the corrugated portion is integrally formed with the body.

4. A device according to claim 1 wherein the body comprises a tubular portion having an axially oriented recess and the corrugated portion is provided within the axially oriented recess.

5. A device according to claim 1 wherein the corrugated portion surrounds the fluid flow path.

6. A device according to claim 1 wherein the corrugated portion has an axial length of between 50 and 300 mm.

7. A device according to claim 1 wherein the fluid flow path is a tubular fluid flow path having a diameter of equal to or greater than 5 mm.

8. (canceled)

9. A device according to claim 1 wherein the device is a patient inhalation/exhalation device comprising:
at least one aperture for inlet or outlet of air into/from the device;
a mouthpiece for communication with the mouth of the patient;
a body defining an air flow path extending between the aperture and the mouthpiece along which air is drawn to the mouthpiece by inhalation by the patient or air is forced towards the aperture by exhalation by the patient, the body comprising an air flow rate indicator operable to alternatively generate at least a first and a second sound signal indicative of the air flow rate along the air flow path in a first direction,
wherein the air flow rate indicator comprises a corrugated portion having a plurality of corrugations extending into the fluid flow path.

10. (canceled)

11. A device for indicating a fluid flow rate, the device comprising:
an aperture;
a mouthpiece; and
a body defining a tubular fluid flow path extending between the aperture and the mouthpiece, the body comprising a fluid flow rate indicator operable to generate a sound signal indicative of the fluid flow rate along the fluid flow path,
wherein the fluid flow rate indicator comprises a corrugated portion having at least one corrugation extending into the fluid flow path, and wherein the tubular fluid flow path has a diameter greater than 8 mm.

12. A device according to claim 11 wherein the body is at least partly formed of plastics material.

13. A device according to claim 11 wherein the corrugated portion is integrally formed with the body.

14. A device according to claim 11 wherein the body comprises a tubular portion having an axially oriented recess and the corrugated portion is provided within the axially oriented recess.

15. A device according to claim 11 wherein the corrugated portion surrounds the fluid flow path.

16. A device according to claim 11 wherein the corrugated portion has an axial length of between 50 and 300 mm.

17. A device according to claim 11 wherein the corrugated portion comprises a plurality of corrugations and the flow rate indicator is operable to alternatively generate at least a first and a second sound signal indicative of the air flow rate along the air flow path in a first direction.

18. A device according to claim 11 wherein the device is a patient inhalation/exhalation device comprising:
at least one aperture for inlet or outlet of air into/from the device;
a mouthpiece for communication with the mouth of the patient;
a body defining a tubular air flow path extending between the aperture and the mouthpiece along which air is drawn to the mouthpiece by inhalation by the patient or air is forced towards the aperture by exhalation by the patient, the body comprising an air flow rate indicator operable to generate a sound signal indicative of the air flow rate along the air flow path,
wherein the air flow rate indicator comprises a corrugated portion having at least one corrugation extending into the fluid flow path, and wherein the tubular fluid flow path has a diameter greater than 8 mm.

19. (canceled)

20. A system comprising:
a device according to claim 1; and
a sound receiver for detecting a sound signal.

21. A method of monitoring peak expiratory or inhalatory flow in a patient, the method comprising:
providing a system according to claim 20,
exhaling or inhaling through the mouthpiece of the device;
detecting the sound signal generated.

22. A method according to claim 21 further comprising comparing the sound signal generated with a reference frequency and providing an alert if the generated sound signal has a lower frequency than the reference frequency.

23. Use of a device according to claim 1 for measuring lung volume or peak inhalatory/exhalatory flow in a patient.

24-27. (canceled)
